(19) United States
(12) Patent Application Publication
(10) Pub. No.: US 2008/0097183 A1
(43) Pub. Date: Apr. 24, 2008

(54) PASSIVE IN VIVO SUBSTANCE SPECTROSCOPY

(76) Inventor: Donald Martin Monro, Beckington (GB)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/470,615

(22) Filed: Sep. 6, 2006

(51) Int. Cl. A61B 5/00

(52) U.S. Cl. 600/407

(57) ABSTRACT

Embodiments of methods, apparatuses, systems and/or devices for passive in vivo substance spectroscopy are disclosed.

### FIELD

This disclosure is related to spectroscopy and, in particular, passive in vivo substance spectroscopy.

### BACKGROUND

In a variety of contexts, having the ability to passively perform in vivo substance spectroscopy may be desirable.

### BRIEF DESCRIPTION OF THE DRAWINGS

Subject matter is particularly pointed out and distinctly claimed in the concluding portion of the specification. Claimed subject matter, however, both as to organization and method of operation, together with objects, features, and advantages thereof, may best be understood by reference of the following detailed description if read with the accompanying drawings in which:

FIG. 1 is a plot illustrating the absorption features of Herring DNA;

FIG. 2 is a plot illustrating the absorption features of Salmon DNA; and

FIG. 3 is a schematic diagram illustrating one embodiment of an apparatus for passive in vivo substance spectroscopy.

### DETAILED DESCRIPTION

In the following detailed description, numerous specific details are set forth to provide a thorough understanding of claimed subject matter. However, it will be understood by those skilled in the art that claimed subject matter may be practiced without these specific details. In other instances, well-known methods, procedures, components and/or circuits have not been described in detail so as not to obscure claimed subject matter.

Some portions of the detailed description which follow are presented in terms of algorithms and/or symbolic representations of operations on data bits and/or binary digital signals stored within a computing system, such as within a computer and/or computing system memory. These algorithmic descriptions and/or representations are the techniques used by those of ordinary skill in the data processing arts to convey the substance of their work to others skilled in the art. An algorithm is here, and generally, considered to be a self-consistent sequence of operations and/or similar processing leading to a desired result. The operations and/or processing may involve physical manipulations of physical quantities. Typically, although not necessarily, these quantities may take the form of electrical and/or magnetic signals capable of being stored, transferred, combined, compared and/or otherwise manipulated. It has proven convenient, at times, principally for reasons of common usage, to refer to these signals as bits, data, values, elements, symbols, characters, terms, numbers, numerals and/or the like. It should be understood, however, that all of these and similar terms are to be associated with appropriate physical quantities and are merely convenient labels. Unless specifically stated otherwise, as apparent from the following discussion, it is appreciated that throughout this specification discussions utilizing terms such as “processing”, “computing”, “calculating”, “determining” and/or the like refer to the actions and/or processes of a computing platform, such as a computer or a similar electronic computing device, that manipulates and/or transforms data represented as physical electronic and/or magnetic quantities and/or other physical quantities within the computing platform's processors, memories, registers, and/or other information storage, transmission, and/or display devices.

In this context, in vivo substance spectroscopy refers to methods of identifying or characterising substances in vivo by measuring data in a form known to vary between different substances so that the capability is provided to a greater or lesser extent of distinguishing between different substances for identification or other purposes. In this context, therefore, the term passive in vivo substance spectroscopy includes the use of passive electromagnetic emissions to distinguish between or identify in vivo substances, such as for identification purposes, for example.

As is well-known, a human has a unique DNA. Despite its simple sequence of bases, the DNA molecule, in effect, codes aspects of a particular species' characteristics. Furthermore, for each individual, it codes unique distinguishing biological characteristics of that individual. The DNA of an individual is also inherited at least partly from a biological parent and may be used to identify the individual or their ancestry. Work has gone on for many years, and is continuing, to relate particular DNA sequences to characteristics of a person having that DNA sequence. Thus, the DNA of an individual may reveal the genes inherited by an individual and may also, in some cases, reveal an abnormality or predisposition to certain inherited diseases, for example.

From the fields of chemistry and physics, atoms and molecules are known to provide a unique response if exposed to electromagnetic radiation, such as radio waves and/or light, for example. At the atomic or molecular level, radiation may be absorbed, reflected, or emitted by the particular atom or molecule. This produces a unique signature, although which of these phenomena take place may vary depending at least in part upon the particular frequency of the radiation impinging upon the particular atom or molecule.

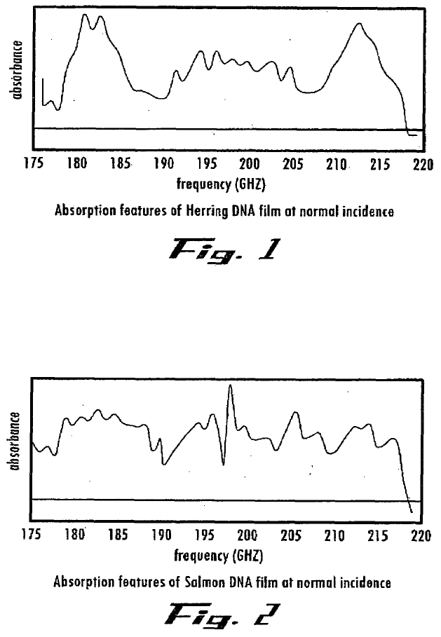

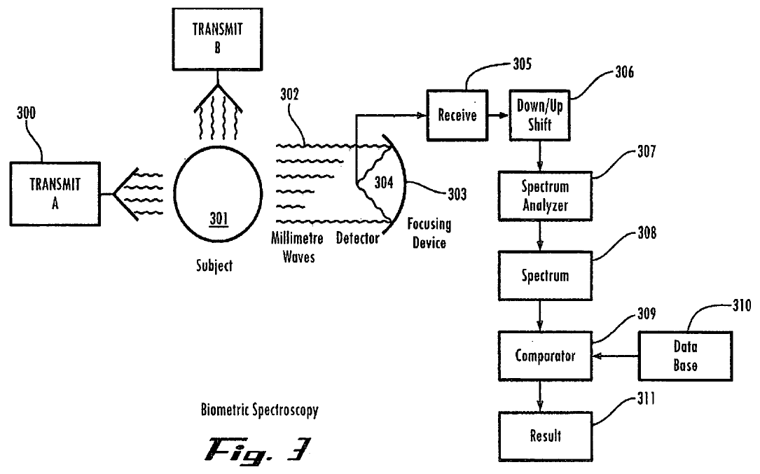

Experiments have shown that species may be distinguished by their absorption spectrum in millimetre electromagnetic waves. See Jing Ju, “Millimeter Wave Absorption Spectroscopy of Biological Polymers,” PhD Thesis, Stevens Institute of Technology, Hoboken, N.J., 2001. For example, FIGS. 1 and 2 illustrate absorption features of Herring and Salmon DNA, respectively. An approach, although claimed subject matter is not limited in scope in this respect, may include applying or observing a range of millimetre wavelengths and recording the spectral response to those millimetre wavelengths at a receiver. In such an approach, peaks and troughs in the spectral response may provide a spectrum or signature for comparison.

Sensitive instruments exist capable of receiving radiation naturally emitted by objects that are warmer than their surroundings or ‘background.’ One example is thermal imaging by enhancing infrared radiation. Likewise, imaging devices capable of producing pictures from emitted millimetre waves exist, such as the Quinetic Borderwatch system, currently being deployed in security systems. It is also noted that Astronomy, either optical or radio, relies on emitted radiation above the background.

Therefore, waves originating within a sample may be detected and/or recorded. Likewise, those waves may be absorbed, scattered or reflected by the sample or the object of the radiation. At certain frequencies, modes of vibration of molecules or atoms in a sample result in radiation at that frequency being more highly absorbed, scattered or reflected compared to waves at other frequencies. At some frequencies, the sample may even emit more energy than it receives by a process that transfers energy to a resonant mode of vibration from an absorptive one.

In one embodiment, naturally emitted waves in the appropriate range may be observed as absorbing and/or emitting resonances in the molecules and structures they encounter as they pass through the body that emits them. A suitably sensitive receiver may be constructed so as to scan a suitable range of frequencies. Such a receiver may therefore detect and likewise may be employed to produce a spectrographic pattern which is characteristic of the structures and/or molecules that encountered the radiation. Due at least in part to differences in molecular structure, different DNA and/or other substances in vivo will produce different spectrographic patterns at the receiver. Therefore, as explained in more detail below, in vivo substances, for example, may be differentiated by a signature spectrum, such as, for example, peaks and troughs in the spectrum, of passively emitted radiation over a suitable range of frequencies.

An advantage of this particular embodiment is that electromagnetic radiation that is emitted naturally and generated passively, in general, presents fewer safety concerns for living tissue, for example, than other approaches. However, claimed subject matter is not limited in scope to this particular advantage, of course.

Although claimed subject matter is not limited in scope in this respect, a wide range of frequencies may potentially be employed. For example, Wien's law tells us that objects of different temperatures emit spectra that peak at different wavelengths. Therefore, at the temperature of the human body, for example, approximately 37 degrees Celsius, Wien's law indicates that the wavelength of maximum emitted radiation is approximately 9×10⁻³ millimetres, or 9 microns. This is a wavelength between conventionally short radio waves and conventionally long light waves. Expressed as frequency, it is about 32 Terra Hertz, although, of course, frequencies above and below this frequency may also be measured. For example, there is a relatively respectable amount of radiation below this being emitted that is capable of being measured, down to, for example, approximately 10 MHz, and perhaps below that.

Applying Plank's law of black body radiation, one may calculate the energy emitted per Hertz of bandwidth from each square centimetre of the body surface as a function of frequency. Although this calculation by itself is not necessarily an indication of the feasibility of detection; nonetheless, it may be converted to obtain the number of quanta of radiation emitted per second. At a frequency of 10 THz, according to Planck's law, the human body emits 6×10⁴ quanta per Hertz of bandwidth from every square centimetre of its surface into every radian of solid angle it faces. At substantially the same frequency, the surroundings of the human body at 20 degrees Celsius also emits ‘background’ radiation, but the human body will emit 6,400 quanta more than the background, again using Planck's law.

The following table gives the approximate numbers of quanta from 10 MHz to 100 THz emitted per square centimetre into a cone of solid angle 1 radian, as calculated from Planck's law. If a wide enough spectral range is considered, spectrographic analysis of the emitted radiation may be performed. Sensitive receivers are able to detect a few quanta. Therefore, spectrographic analysis of emitted radiation may be performed by measuring a sufficiently wide enough spectral range, such as, for example, from below 10 MHz to over 32 THz, sufficient quanta may be obtained to form a spectrogram.

| Frequency | | Wavelength | Quanta/Hz |
|---|---|---|---|
| 100 | MHz | 3 meters | .1 |
| 1 | GHz | 30 cm | 14 |
| 10 | GHz | 3 cm | 140 |
| 100 | GHz | .3 cm (3 mm) | 1400 |
| 1 | THz | .3 mm | 13,000 |
| 10 | THz | .03 mm (30 microns) | 60,000 |
| 100 | THz | 3 microns | 4 |

A receiver may be made directional to collect quanta from a warm body, such as a human, for example, so that more than 1 square centimetre is sensed. For example, focusing radiation using a reflector, as shown in FIG. 3, or by some other method may be employed.

Referring to FIG. 3, for example, subject 301 may passively emit millimeter waves 302 which are focused by a focusing device 305 onto a detector 304. Signals from detector 304 may be passed to a receiver 305 which may amplify the signals before down-shifting or up-shifting the signals, at 306, to a frequency range convenient for spectrum analyzer 307. Spectrum analyzer 307 may operate in a radio frequency or optical range, whichever may be convenient for the frequency range of interest. Resulting spectrum 308 may be compared, at 309, with previously stored spectrograms, such as, in this example, from a database 310, to produce a result 311 indicative of the quality of the match between spectrum 308 for subject 301 and spectra from database 310. Of course, this is merely one example embodiment provided for purposes of illustration. Many other embodiments are possible and are included within the scope of claimed subject matter.

It is possible that any of the frequencies mentioned above might be used and claimed subject matter is intended to cover such frequencies mentioned; however, one range to be employed, for example, may be from approximately 10 GHz to approximately 1 THz, although, again, claimed subject matter is not limited in scope in this respect. The range to 32 THz and above may be attractive from the number of quanta emitted. Of course, it is difficult to predict how developments in technology may affect or influence an appropriate frequency range for use in such an application. Nonetheless, this interestingly corresponds with a prediction made by Van Zandt and Saxena in 1988, that some DNA molecules may be expected to exhibit resonances in approximately this range. See Van Zandt and Saxena, “Millimetre-microwave spectrum of DNA: Six predictions for Spectroscopy,” Phys. Rev. A 39, No. 5, pp 2692-2674, March, 1989. Likewise, a recent finding by Jing Ju indicates DNA from various species of fish and bacteria may be differentiated by millimetre wave spectroscopy in the range of approximately 180 to approximately 220 GHz. See Jing Ju, “Millimeter Wave Absorption Spectroscopy of Biological Polymers,” PhD Thesis, Stevens Institute of Technology, Hoboken, N.J., 2001.

For example, for one embodiment of claimed subject matter, it may be possible to detect substances by characteristic peaks and troughs in a spectrum of passive emitted radiation over a suitable range of frequencies. Typically, this particular embodiment will not match all peaks and troughs of the spectrum. Instead, this particular embodiment should examine a spectrum for peaks and troughs that are characteristic of a substance that is being sought. These will, in general, be mixed with peaks and troughs characteristic of other substances, for example, but a priori knowledge of the spectrum of a particular substance will enable this particular embodiment, for example, to seek a particular spectrum for a particular substance. It will, of course, occur that peaks and troughs not belonging to the substance in question may occur close to or at the same frequencies of the substance of interest. However, if resolution is sufficiently fine, obscuration of a peak or trough of interest is in general not a significant issue. A complex molecule should, for example have many characteristic peaks and troughs, so by looking for a sufficient number of characteristic peaks and troughs over a sufficiently wide range of frequencies, detection may be confirmed to a high degree of statistical certainty as any person versed in statistics will know. For some embodiments, as described in more detail hereinafter, potential feature relates to detecting differences between spectrographs.

A variety of potential applications of an embodiment, such as the one just described are possible and contemplated. However, claimed subject matter is not limited in scope to this embodiment or to these applications. Many other potential embodiments and many other applications are envisioned. Nonetheless, here we provide a few examples of illustrative applications. For example, in sport, concerted efforts are made at great expense to prevent participants from cheating by using substances that enhance performance in competition or provide an advantage if otherwise undetected. Furthermore, this may extend beyond humans, as a similar problem arises in other sporting areas, such as racing of horses, or raising dogs, to provide a few examples.

Another potential application includes medicine. Here, it would be desirable for a substance to be detected and have its concentration measured by this method. A simple non-invasive test in which an individual stands in front of a passive millimetre wave or infrared spectrograph and a desired substance is be detected would be useful in human and veterinary medicine.

In an alternate embodiment, it may be desirable to have the capability to detect a change in a spectrograph taken on separate occasions. As mentioned previously, in such an embodiment, detecting differences between spectrographs may provide valuable for such embodiments. For example, this might be indicative of the presence of a substance in one sample, but not another, as an example. This may prove useful in many areas. In medicine, a change in biochemistry of an individual, for example, may be indicative of the appearance of a disease. By this method, it is hoped to provide the ability to screen non-invasively and potentially inexpensively for a standard range of substances, hopefully a range that is more extensive than is currently achieved through blood or urine sampling. Furthermore, after an individual, for example, has his or her characteristic biochemical spectrum registered in a system, it will be possible to monitor changes and detect a wide range of substances and conditions, even without an a priori suspected diagnosis, and without knowing what a substance is the first time it is detected.

Returning to potential implementation issues for a particular embodiment, it is noted that, in some situations, an emitting body may not be much warmer than its surroundings, so that long measurements may be desirable to obtain sufficient quanta to get a reasonable resolution of the spectrogram. In such situations, it may also be desirable to take steps to reduce measurement time. Any one of a number of techniques may be employed if this is desired. For example, one approach may be to place the individual in a suitable environment in which the background emits the radiation of a cold body. In another approach, radiation may be focused on a detector to increase its intensity, including large reflectors that at least partly or wholly surround the subject. Likewise, both approaches may be employed in some embodiments, if desired. In yet another approach, measurement time may be reduced by employing multiple receivers. For example, in one such embodiment, different receivers may be employed to cover different parts of the spectrum, such as a case in which some receivers are optical receivers and others are radio receivers, although, of course, claimed subject matter is not limited in scope in this respect.

Likewise, a variety of spectrographic and detection techniques could be employed. In one embodiment, radio waves could be sampled and Analog-to-Digital (A/D) conversion may be employed, either directly at lower frequencies, or after modulation by a suitable carrier for down conversion to lower frequencies. In this embodiment, spectral analysis may be accomplished by applying well-known Fast Fourier Transform (FFT) techniques, for example. In such an embodiment, sampling rate and sampling duration are parameters that may affect bandwidth and line width, respectively.

In another embodiment, the frequency of the waves may be modulated upwards by an optical carrier into the optical or infra-red range and spectral analysis may be accomplished through application of standard optical spectrographic techniques, such as application of prism or prism-like technology so that light of different frequencies may be focused to detectors corresponding to a particular light frequency. Frequencies characteristic of an individual may also be related to characteristics that differentiate the absorption or radiation characteristics of an individual, in addition to or instead of DNA resonances, depending on the particular embodiment, for example. Therefore, the range of frequencies to be employed may vary. Furthermore, claimed subject matter is not limited in scope to a particular range, of course.

It will, of course, be understood that, although particular embodiments have just been described, claimed subject matter is not limited in scope to a particular embodiment or implementation. For example, one embodiment may be in hardware, such as implemented to operate on a device or combination of devices, for example, whereas another embodiment may be in software. Likewise, an embodiment may be implemented in firmware, or as any combination of hardware, software, and/or firmware, for example. Likewise, although claimed subject matter is not limited in scope in this respect, one embodiment may comprise one or more articles, such as a storage medium or storage media. This storage media, such as, one or more CD-ROMs and/or disks, for example, may have stored thereon instructions, that if executed by a system, such as a computer system, computing platform, or other system, for example, may result in an embodiment of a method in accordance with claimed subject matter being executed, such as one of the embodiments previously described, for example. As one potential example, a computing platform may include one or more processing units or processors, one or more input/output devices, such as a display, a keyboard and/or a mouse, and/or one or more memories, such as static random access memory, dynamic random access memory, flash memory, and/or a hard drive.

In the preceding description, various aspects of claimed subject matter have been described. For purposes of explanation, specific numbers, systems and/or configurations were set forth to provide a thorough understanding of claimed subject matter. However, it should be apparent to one skilled in the art having the benefit of this disclosure that claimed subject matter may be practiced without the specific details. In other instances, well known features were omitted and/or simplified so as not to obscure claimed subject matter. While certain features have been illustrated and/or described herein, many modifications, substitutions, changes and/or equivalents will now occur to those skilled in the art. It is, therefore, to be understood that the appended claims are intended to cover all such modifications and/or changes as fall within the true spirit of claimed subject matter.

1. A method comprising:
passively measuring electromagnetic radiation in the vicinity of an in vivo substance over a range of frequencies to obtain a frequency spectrum; and
comparing the obtained frequency spectrum to one or more frequency spectra signatures.

2. The method of claim 1, and further comprising;
identifying the frequency spectrum of the one or more frequency spectra that most closely resembles the obtained frequency spectrum.

3. The method of claim 2, wherein identifying the one or more frequency spectra that most closely resemble the obtained frequency spectrum distinguishes between different in vivo substances

4. The method of claim 1, wherein the electromagnetic radiation falls in the range from approximately 10 GHz to approximately 1 THz.

5. The method of claim 1, wherein electromagnetic radiation is passively measured for a sufficiently long period of time to obtain sufficient quanta to produce a spectrogram.

6. The method of claim 1, wherein electromagnetic radiation is passively measured by focusing the radiation.

7. The method of claim 1, wherein electromagnetic radiation is passively measured by employing multiple detectors.

8. The method of claim 7, wherein different detectors cover a different range of frequencies.

9. An apparatus comprising:
a detector to passively measure electromagnetic radiation in the vicinity of an in vivo substance over a range of frequencies;
a device to produce a spectrogram from the detector measurements; and
a computing platform adapted to compare said spectrogram against other spectra to be stored on said computing platform.

10. The apparatus of claim 9, wherein said detector includes a mechanism to focus said electromagnetic radiation for measurement.

11. The apparatus of claim 9, wherein said device to produce said spectrogram includes an A/D converter and is capable of implementing an FFT.

12. The apparatus of claim 9, wherein said device to produce said spectrogram is adapted to shift the range of frequencies for spectrum analysis.

13. An apparatus comprising:
means for passively measuring electromagnetic radiation in the vicinity of an in vivo substance over a range of frequencies to obtain a frequency spectrum; and
means for comparing the obtained frequency spectrum to one or more frequency spectra signatures.

14. The apparatus of claim 13, and further comprising;
means for identifying the frequency spectrum of the one or more frequency spectra that most closely resembles the obtained frequency spectrum.

15. The apparatus of claim 14, wherein means for identifying the one or more frequency spectra that most closely resemble the obtained frequency spectrum comprises means for distinguishing between different in vivo substances.

16. The apparatus of claim 13, wherein the electromagnetic radiation falls in the range from approximately 10 GHz to approximately 1 THz.

17. An article comprising: a storage medium having stored thereon instructions that, if executed, result in execution of the following method by a computing platform:
passively measuring electromagnetic radiation in the vicinity of an in vivo substance over a range of frequencies to obtain a frequency spectrum; and
comparing the obtained frequency spectrum to one or more frequency spectra signatures.

18. The article of claim 17, wherein said storage medium comprises instructions that, if executed, further results in identifying the frequency spectrum of the one or more frequency spectra that most closely resembles the obtained frequency spectrum.

19. The article of claim 18, wherein said storage medium comprises instructions that, if executed, further results in identifying the one or more frequency spectra that most closely resemble the obtained frequency spectrum distinguishes between different in vivo substances.

20. The article of claim 17, wherein said storage medium comprises instructions that, if executed, further results in wherein electromagnetic radiation is passively measured for a sufficiently long period of time to obtain sufficient quanta to produce a spectrogram.

21. A method comprising:
passively measuring electromagnetic radiation in the vicinity of an in vivo substance over a range of frequencies to obtain a frequency spectrum; and
comparing the obtained frequency spectrum with one or more frequency spectra signatures to identify differences between spectra.

22. The method of claim 21, wherein said one or more frequency spectra comprise one or more spectra of the in vivo substance measured at one or more times other than the time said frequency spectrograph was measured.

23. The method of claim 21, wherein the electromagnetic radiation falls in the range from approximately 10 GHz to approximately 1 THz.

24. The method of claim 21, wherein electromagnetic radiation is passively measured for a sufficiently long period of time to obtain sufficient quanta to produce a spectrogram.

25. The method of claim 21, wherein electromagnetic radiation is passively measured by focusing the radiation.

26. The method of claim 21, wherein electromagnetic radiation is passively measured by employing multiple detectors.

27. The method of claim 26, wherein different detectors cover a different range of frequencies.

28. An apparatus comprising:
a detector to passively measure electromagnetic radiation in the vicinity of an in vivo substance over a range of frequencies;
a computing platform adapted to produce a spectrogram from the detector measurements and to compare said spectrogram against other spectra to be stored on said computing platform to identify differences between spectra.

29. The apparatus of claim 28, wherein said other spectra comprise were measured at times other than the time the detector measurements were measured to produce said spectrograph.

30. An article comprising: a storage medium having stored thereon instructions that, if executed, result in execution of the following method by a computing platform:
passively measuring electromagnetic radiation in the vicinity of an in vivo substance over a range of frequencies to obtain a frequency spectrum; and
comparing the obtained frequency spectrum with one or more frequency spectra signatures to identify differences between said spectrum and said one or more spectra.

31. The article of claim 30, wherein said instructions if executed further result in said one or more frequency spectra comprising detector measurements for said in vivo substance made at times other than the detector measurements used to produce said spectrograph.
